(19) United States
(12) Patent Application Publication
(10) Pub. No.: US 2023/0386665 A1
(43) Pub. Date: Nov. 30, 2023

(54) METHOD AND DEVICE FOR CONSTRUCTING AUTISM SPECTRUM DISORDER (ASD) RISK PREDICTION MODEL

(71) Applicant: SUN YAT-SEN UNIVERSITY, Guangzhou (CN)

(72) Inventors: Jin Jing, Guangzhou (CN); Xiuhong Li, Guangzhou (CN); Jiajie Chen, Guangzhou (CN); Xin Wang, Guangzhou (CN); Lizi Lin, Guangzhou (CN); Muqing Cao, Guangzhou (CN); Ning Pan, Guangzhou (CN); Xiujin Lin, Guangzhou (CN); Hailin Li, Guangzhou (CN); Jingjing Zeng, Guangzhou (CN); Siyu Liu, Guangzhou (CN); Xiaoling Zhan, Guangzhou (CN); Chengkai Jin, Guangzhou (CN); Shuolin Pan, Guangzhou (CN)

(21) Appl. No.: 18/232,363

(22) Filed: Aug. 10, 2023

Related U.S. Application Data
(63) Continuation-in-part of application No. PCT/CN2022/120423, filed on Sep. 22, 2022.

(30) Foreign Application Priority Data
Oct. 11, 2021 (CN) 202111182323.3

(51) Int. Cl. G16H 50/20; G16H 50/30; G06N 20/20

(52) U.S. Cl. CPC G16H 50/20; G16H 50/30; G06N 20/20

(57) ABSTRACT

The present disclosure provides a method and device for constructing an autism spectrum disorder (ASD) risk prediction model. The method includes: establishing a first data table and a second data table based on case information of a sample set, obtaining a first grouped table set and a second grouped table set according to a preset characteristic arrangement rule and marker grouping rule, training data based on a random forest machine learning algorithm, and importing test data to obtain a first best characteristic combination and a second characteristic combination; and obtaining a first model based on the first best characteristic combination, stratified sampling of the first data table, and the random forest machine learning algorithm, obtaining a second model based on the second best characteristic combination, stratified sampling of the second data table, and the random forest machine learning algorithm, and performing combination to construct an ASD risk prediction model.

### CROSS REFERENCE TO RELATED APPLICATIONS

The present application is a Continuation-In-Part Application of PCT Application No. PCT/CN2022/120423 filed on Sep. 22, 2022, which claims the benefit of Chinese Patent Application No. 202111182323.3 filed on Oct. 11, 2021. All the above are hereby incorporated by reference in their entirety.

### TECHNICAL FIELD

The present disclosure relates to the field of autism spectrum disorder (ASD) risk prediction, and in particular, to a method and device for constructing an ASD risk prediction model.

### BACKGROUND

As a group of severe neurodevelopmental disorders, ASD is mainly characterized by core symptoms such as social communication disability and narrow/repetitive interest or behavior. At present, ASD is still diagnosed mainly by performing clinical observation by doctor, collecting a growth and development history, making a mental examination, and evaluating a degree of a child's symptom based on various screening and symptom evaluation scales, such as eye tracking technology and brain magnetic resonance imaging technology.

However, at represent, a result of evaluating the degree of the symptom of a child varies from person to person, and there is no unified standard. In manual evaluation, in order to obtain an accurate evaluation result, high professional and empirical requirements are imposed on an evaluator, resulting in a very high labor cost. Most of existing ASD risk prediction models have many evaluation items, take too long time, and the like, resulting in a large error and inaccurate prediction data.

Therefore, those skilled in the art urgently need a high-accuracy prediction model that can process a result of an ASD evaluation item and obtain predictive data and results.

### SUMMARY

A technical problem to be resolved in the present disclosure is to provide a method and device for constructing an ASD risk prediction model, to effectively improve efficiency of processing a result of an ASD evaluation item and accuracy of obtained prediction data in the prior art.

In order to resolve the above technical problem, the present disclosure provides a method for constructing an ASD risk prediction model, including:
establishing a first data table and a second data table based on case information of a sample set, where the sample set includes a sample of a mild to moderate ASD case, a sample of a severe ASD case, and a sample of a normal case, the first data table records case information of the sample of the normal case and case information of samples of all ASD cases, the second data table records case information of the sample of the mild to moderate ASD case and case information of the sample of the severe ASD case, and each piece of case information includes a characteristic, a characteristic variable, and a marker;
performing characteristic arrangement and marker grouping on the first data table and the second data table according to a preset characteristic arrangement rule and marker grouping rule to obtain a first grouped table set and a second grouped table set respectively, where the first grouped table set includes a first test table set and a first training table set, the second grouped table set includes a second test table set and a second training table set;
training and modeling the first training table set and the second training table set based on a random forest machine learning algorithm to obtain a first submodel set and a second submodel set respectively, importing the first test table set into the first submodel set to obtain a first best characteristic combination, and importing the second test table set into the second submodel set to obtain a second best characteristic combination;
obtaining a first model based on the first best characteristic combination, stratified sampling of the first data table, and the random forest machine learning algorithm, obtaining a second model based on the second best characteristic combination, stratified sampling of the second data table, and the random forest machine learning algorithm, and combining the first model and the second model to construct an ASD risk prediction model, so as to input a result of an ASD evaluation item into the ASD risk prediction model to obtain a prediction result.

Further, the establishing a first data table and a second data table based on case information of a sample set specifically includes:
based on the sample of the mild to moderate ASD case, the sample of the severe ASD case, and the sample of the normal case in the sample set, collecting and preprocessing data information of the ASD evaluation item, extracting a general characteristic, a characteristic variable, and a marker of the sample, screening out a common characteristic variable, calculating a score of each characteristic variable in ASD test indicator data information according to a preset scoring method, screening out a characteristic variable that can reflect a score of the ASD test indicator data information, and establishing the first data table and the second data table.

Further, the performing characteristic arrangement on the first data table and the second data table separately according to a preset characteristic arrangement rule specifically includes:
calculating a weight value of each characteristic in the data table according to a preset characteristic weight calculation method, sorting the corresponding characteristic based on the weight value of each characteristic, and performing characteristic extraction and addition on a characteristic-sorted first data table and a characteristic-sorted second data table to obtain a first sequence table set and a second sequence table set respectively, where
the performing characteristic extraction and addition on a characteristic-sorted first data table and a characteristic-sorted second data table specifically includes:
extracting the first two characteristics from the characteristic-sorted first data table and the characteristic-sorted second data table based on a characteristic arrangement order to form a first subsequence table and a second subsequence table respectively, then sequentially adding a next characteristic to the first subsequence table and the second subsequence table based on the characteristic arrangement order until all characteristics in the first data table and the second data table are added, to obtain a plurality of first subsequence tables and a plurality of second subsequence tables respectively, and combining the plurality of first subsequence tables and the plurality of second subsequence tables to obtain the first sequence table set and the second sequence table set respectively.

Further, the performing marker grouping on the first data table and the second data table according to a preset marker grouping rule to obtain a first grouped table set and a second grouped table set specifically includes:
performing stratified marker sampling on all first subsequence tables in the first sequence table set and all second subsequence tables in the second sequence table set based on a preset table marker grouping condition and a same proportion of evenly divided markers to obtain the first grouped table set and the second grouped table set respectively.

Further, the training and modeling the first training table set and the second training table set based on a random forest machine learning algorithm to obtain a first submodel set and a second submodel set respectively, importing the first test table set into the first submodel set to obtain a first best characteristic combination, and importing the second test table set into the second submodel set to obtain a second best characteristic combination specifically includes:
training and modeling the first training table set and the second training table set based on the random forest machine learning algorithm to obtain the first submodel set and the second submodel set respectively;
importing data of the first test table set into the first submodel set to obtain a corresponding sensitivity and specificity of each first submodel, performing mean value summation to obtain a characteristic combination in a first submodel corresponding to a maximum sum of the sensitivity and the specificity, and taking the obtained characteristic combination as the first best characteristic combination; and
importing data of the second test table set into the second submodel set to obtain a corresponding sensitivity and specificity of each second submodel, performing mean value summation to obtain a characteristic combination in a second submodel corresponding to a maximum sum of the sensitivity and the specificity, and taking the obtained characteristic combination as the second best characteristic combination.

Further, the obtaining a first model based on the first best characteristic combination, stratified sampling of the first data table, and the random forest machine learning algorithm, and obtaining a second model based on the second best characteristic combination, stratified sampling of the second data table, and the random forest machine learning algorithm specifically includes:
performing, based on the first best characteristic combination, the stratified sampling on a characteristic that meets the first best characteristic combination in the first data table, and performing, based on the random forest machine learning algorithm, iterative operation on a first data table obtained after the stratified sampling to obtain the first model; and
performing, based on the second best characteristic combination, the stratified sampling on a characteristic that meets the second best characteristic combination in the second data table, and performing, based on the random forest machine learning algorithm, the iterative operation on a second data table obtained after the stratified sampling to obtain the second model.

Further, the combining the first model and the second model to construct an ASD risk prediction model, so as to input a result of an ASD evaluation item into the ASD risk prediction model to obtain a prediction result specifically includes:
extracting one test sample from the first data table obtained after the stratified sampling and the second data table obtained after the stratified sampling, and inputting data information that meets the first best characteristic combination in the test sample into the first model to obtain a first predicted probability of the test sample, where the first predicted probability includes a total predicted probability of an ASD case and a predicted probability of the normal case;
if the total predicted probability of the ASD case is less than the predicted probability of the normal case, determining that the test sample is a normal case; or if the total predicted probability of the ASD case is greater than the predicted probability of the normal case, inputting data information that meets the second best characteristic combination in the test sample into the second model to obtain a second predicted probability of the test sample, where the second predicted probability includes a predicted probability of the mild to moderate ASD case and a predicted probability of the severe ASD case;
if the predicted probability of the mild to moderate ASD case is greater than the predicted probability of the severe ASD case, determining that the test sample is a mild to moderate ASD case; or if the predicted probability of the mild to moderate ASD case is less than the predicted probability of the severe ASD case, determining that the test sample is a severe ASD case; and
if the determining result is consistent with an actual situation of the test sample, combining the first model and the second model to construct the ASD risk prediction model, so as to input the result of the ASD evaluation item into the ASD risk prediction model to obtain the prediction result.

In addition, the present disclosure further provides a device for constructing an ASD risk prediction model, including: a data table establishment module, a data sorting module, a characteristic extraction module, and a model construction module, where
the data table establishment module is configured to establish a first data table and a second data table based on case information of a sample set, where the sample set includes a sample of a mild to moderate ASD case, a sample of a severe ASD case, and a sample of a normal case, the first data table records case information of the sample of the normal case and case information of samples of all ASD cases, the second data table records case information of the sample of the mild to moderate ASD case and case information of the sample of the severe ASD case, and each piece of case information comprises a characteristic, a characteristic variable, and a marker;
the data sorting module is configured to perform characteristic arrangement and marker grouping on the first data table and the second data table according to a preset characteristic arrangement rule and marker grouping rule to obtain a first grouped table set and a second grouped table set respectively, where the first grouped table set includes a first test table set and a first training table set, and the second grouped table set includes a second test table set and a second training table set:
the characteristic extraction module is configured to train and model the first training table set and the second training table set based on a random forest machine learning algorithm to obtain a first submodel set and a second submodel set respectively, import the first test table set into the first submodel set to obtain a first best characteristic combination, and import the second test table set into the second submodel set to obtain a second best characteristic combination; and
the model construction module is configured to: obtain a first model based on the first best characteristic combination, stratified sampling of the first data table, and the random forest machine learning algorithm, obtain a second model based on the second best characteristic combination, stratified sampling of the second data table, and the random forest machine learning algorithm, and combine the first model and the second model to construct an ASD risk prediction model, so as to input a result of an ASD evaluation item into the ASD risk prediction model to obtain a prediction result.

Further, that the characteristic arrangement and marker grouping are performed on the first data table and the second data table according to the preset characteristic arrangement rule and marker grouping rule to obtain the first grouped table set and the second grouped table set respectively specifically includes following operations:
calculating a weight value of each characteristic in the data table according to a preset characteristic weight calculation method, sorting the corresponding characteristic based on the weight value of each characteristic, and performing characteristic extraction and addition on a characteristic-sorted first data table and a characteristic-sorted second data table to obtain a first sequence table set and a second sequence table set respectively, where the performing characteristic extraction and addition on a characteristic-sorted first data table and a characteristic-sorted second data table specifically includes: extracting the first two characteristics from the characteristic-sorted first data table and the characteristic-sorted second data table based on a characteristic arrangement order to form a first subsequence table and a second subsequence table respectively, then sequentially adding a next characteristic to the first subsequence table and the second subsequence table based on the characteristic arrangement order until all characteristics in the first data table and the second data table are added, and to obtain a plurality of first subsequence tables and a plurality of second subsequence tables respectively, and combining the plurality of first subsequence tables and the plurality of second subsequence tables to obtain the first sequence table set and the second sequence table set respectively; and
performing stratified marker sampling on all first subsequence tables in the first sequence table set and all second subsequence tables in the second sequence table set based on a preset table marker grouping condition and a same proportion of evenly divided markers to obtain the first grouped table set and the second grouped table set respectively.

Further, the training and modeling the first training table set and the second training table set based on a random forest machine learning algorithm to obtain a first submodel set and a second submodel set respectively, importing the first test table set into the first submodel set to obtain a first best characteristic combination, and importing the second test table set into the second submodel set to obtain a second best characteristic combination specifically includes:
training and modeling the first training table set and the second training table set based on the random forest machine learning algorithm to obtain the first submodel set and the second submodel set respectively;
importing data of the first test table set into the first submodel set to obtain a corresponding sensitivity and specificity of each first submodel, performing mean value summation to obtain a characteristic combination in a first submodel corresponding to a maximum sum of the sensitivity and the specificity, and taking the obtained characteristic combination as the first best characteristic combination; and
importing data of the second test table set into the second submodel set to obtain a corresponding sensitivity and specificity of each second submodel, performing mean value summation to obtain a characteristic combination in a second submodel corresponding to a maximum sum of the sensitivity and the specificity, and taking the obtained characteristic combination as the second best characteristic combination.

The following advantageous effects are achieved by implementing the embodiments of the present disclosure:

A method and device for constructing an ASD risk prediction model provided in the present disclosure take a plurality of ASD evaluation items as characteristic information data, and sort and group the data, such that a trained model can resolve problems such as many evaluation items and a long time consumption in an existing ASD risk prediction model, efficiently and accurately process result data of the evaluation items to provide a complete hierarchical result prediction, and finally perform model combination and testing to further improve the accuracy of a prediction result output by the risk prediction model.

### BRIEF DESCRIPTION OF THE DRAWINGS

FIG. 1 is a schematic flowchart of a method for constructing an ASD risk prediction model according to an embodiment of the present disclosure;

FIG. 2 is a flowchart of constructing a first sequence table set in a method for constructing an ASD risk prediction model according to an embodiment of the present disclosure;

FIG. 3 is a flowchart of constructing a second sequence table set in a method for constructing an ASD risk prediction model according to an embodiment of the present disclosure;

FIG. 4 is a flowchart of constructing a first grouped table set in a method for constructing an ASD risk prediction model according to an embodiment of the present disclosure;

FIG. 5 is a flowchart of constructing a second grouped table set in a method for constructing an ASD risk prediction model according to an embodiment of the present disclosure;

FIG. 6 is a flowchart of constructing a first best characteristic combination in a method for constructing an ASD risk prediction model according to an embodiment of the present disclosure;

FIG. 7 is a flowchart of constructing a second best characteristic combination in a method for constructing an ASD risk prediction model according to an embodiment of the present disclosure;

FIG. 8 a flowchart of constructing a first model and a second model in a method for constructing an ASD risk prediction model according to an embodiment of the present disclosure; and

FIG. 9 is a structural diagram of a device for constructing an ASD risk prediction model according to an embodiment of the present disclosure.

### DETAILED DESCRIPTION

To make the objectives, technical solutions, and advantages of the present disclosure clearer, the following describes the technical solutions of the present disclosure in more detail with reference to the accompanying drawings in the present disclosure. Apparently, the described embodiments are merely some rather than all of the embodiments of the present disclosure, and are not intended to limit the present disclosure. All other embodiments obtained by a person of ordinary skill in the art based on the embodiments of the present disclosure without creative efforts shall fall within the protection scope of the present disclosure.

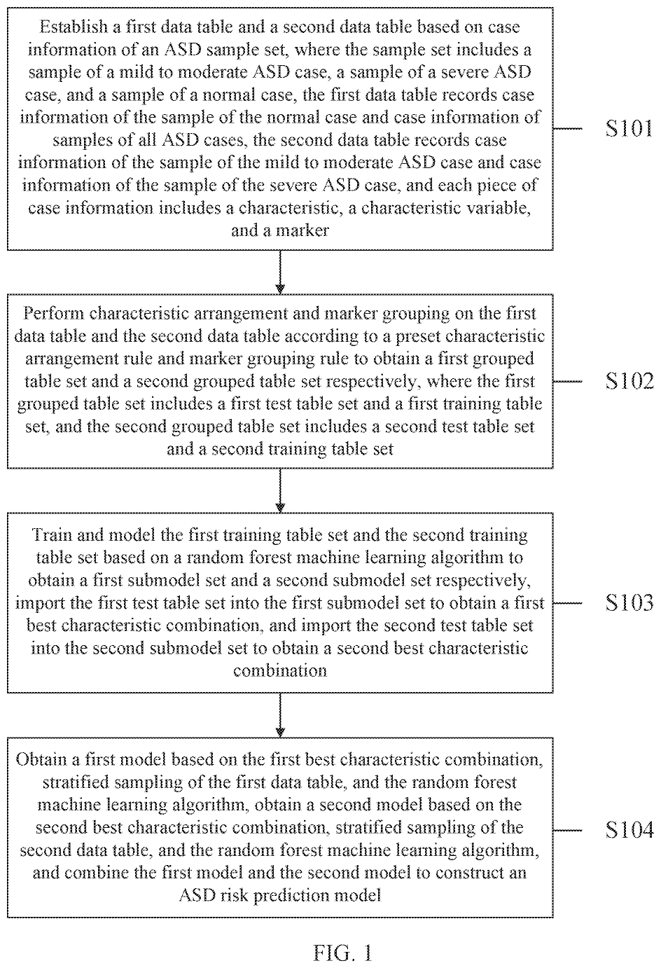

FIG. 1 is a flowchart of a method for constructing an ASD risk prediction model according to an embodiment of the present disclosure. The method includes the following steps:

Step S101: Establish a first data table and a second data table based on case information of a sample set, where the sample set includes a sample of a mild to moderate ASD case, a sample of a severe ASD case, and a sample of a normal case, the first data table records case information of the sample of the normal case and case information of samples of all ASD cases, the second data table records case information of the sample of the mild to moderate ASD case and case information of the sample of the severe ASD case, and each piece of case information includes a characteristic, a characteristic variable, and a marker.

Preferably, in this embodiment, based on 120 mild to moderate ASD cases, 89 severe ASD cases, and 186 normal cases in the sample set, data information of an ASD evaluation item is collected and preprocessed. The data information of the ASD evaluation item includes but is not limited to a demographic characteristic, a common ASD symptom evaluation scale, a lifestyle, and an emotional state.

Preferably, in this embodiment, based on the data information of the ASD evaluation item, a characteristic, a characteristic variable, and a marker of the sample are extracted, a total of 509 common characteristic variables are screened out, a score of each characteristic variable in ASD test indicator data information is calculated according to a preset scoring method, 28 characteristic variables that can reflect a score of the ASD test indicator data information are screened out, and a sample with invalid data is eliminated. A total of 251 cases including 139 normal cases, 72 mild to moderate ASD cases, and 40 severe ASD cases are finally selected for data analysis, to establish the first data table and the second data table by taking the characteristic as a table column, the marker as a table row, and the characteristics variable as a table value.

Preferably, the preset scoring method uses a standard score of the ASD evaluation item as a reference to compare and calculate a score of an actual evaluation item of the sample.

Step S102: Perform characteristic arrangement and marker grouping on the first data table and the second data table according to a preset characteristic arrangement rule and marker grouping rule to obtain a first grouped table set and a second grouped table set respectively, where the first grouped table set includes a first test table set and a first training table set, and the second grouped table set includes a second test table set and a second training table set.

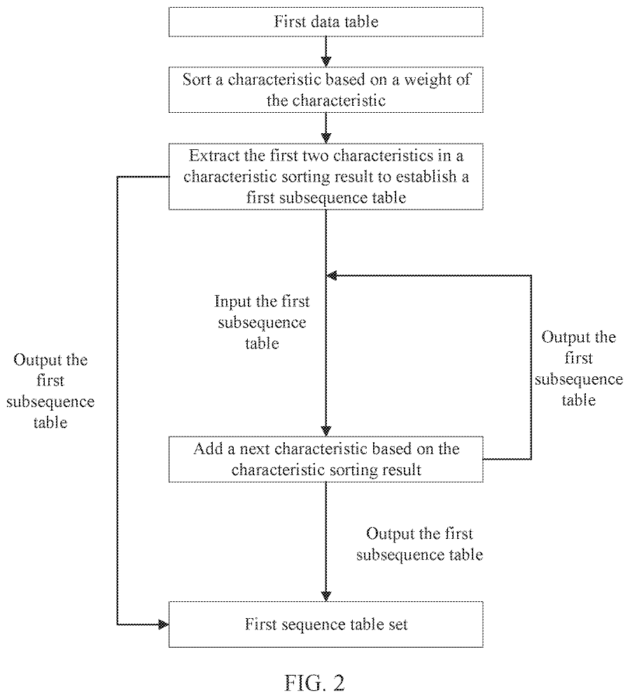

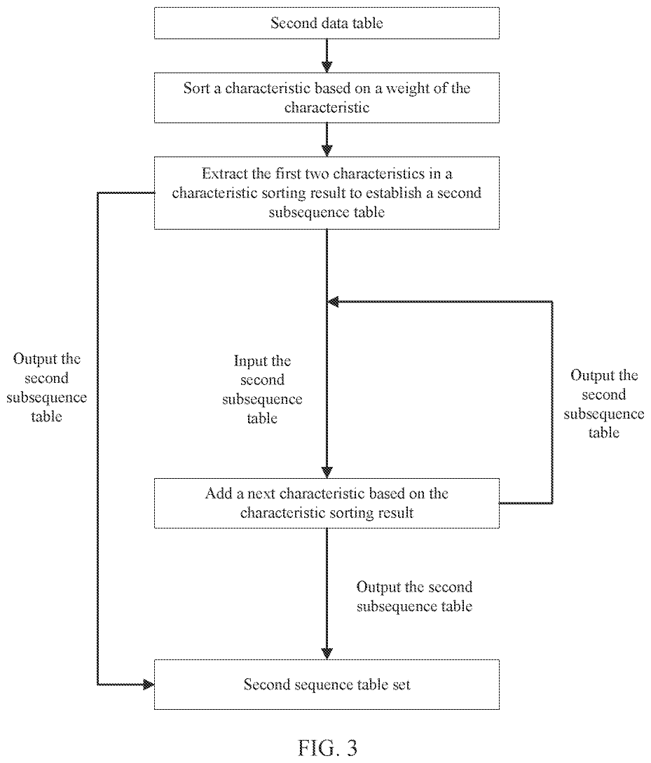

Preferably, as shown in FIG. 2 and FIG. 3, a weight value of each characteristic in the data table is calculated according to a preset characteristic weight calculation method, the corresponding characteristic is sorted based on the weight value of each characteristic, and characteristic extraction and addition are performed on a characteristic-sorted first data table and a characteristic-sorted second data table to obtain a first sequence table set and a second sequence table set respectively.

In this embodiment, as shown in FIG. 2, 28 characteristics and their markers in the first data table are put into a random forest machine learning algorithm, and weight values of the 28 characteristics are obtained by taking a classification accuracy rate as a basis for characteristic importance sorting and according to a characteristic weight calculation method, and are arranged in descending order. As shown in FIG. 3, 28 characteristics and their markers in the second data table are put into the random forest machine learning algorithm, and importance weights of the 28 characteristics are obtained by taking the classification accuracy rate as the basis for characteristic importance sorting, and are arranged in the descending order.

Preferably, as shown in FIG. 2 and FIG. 3, that characteristic extraction and addition are performed on a characteristic-sorted first data table and a characteristic-sorted second data table specifically includes the following operations: extracting the first two characteristics from the characteristic-sorted first data table and the characteristic-sorted second data table based on a characteristic arrangement order to form a first subsequence table and a second subsequence table respectively, then sequentially adding a next characteristic to the first subsequence table and the second subsequence table based on the characteristic arrangement order until all characteristics in the first data table and the second data table are added, to obtain a plurality of first subsequence tables and a plurality of second subsequence tables respectively, and combining the plurality of first subsequence tables and the plurality of second subsequence tables to obtain the first sequence table set and the second sequence table set respectively.

In this embodiment, as shown in FIG. 2, there are a total of 27 first subsequence tables in the first sequence table set. First subsequence table 1 has two characteristics, first subsequence table 2 has three characteristics, . . . , and first subsequence table 27 has 28 characteristics. As shown in FIG. 3, there are a total of 27 second subsequence tables in the second sequence table set. Second subsequence table 1 has two characteristics, second subsequence table 2 has three characteristics, . . . , and second subsequence table 27 has 28 characteristics.

Preferably, stratified marker sampling is performed on all first subsequence tables in the first sequence table set and all second subsequence tables in the second sequence table set based on a preset table marker grouping condition and a same proportion of evenly divided markers to obtain the first grouped table set and the second grouped table set respectively.

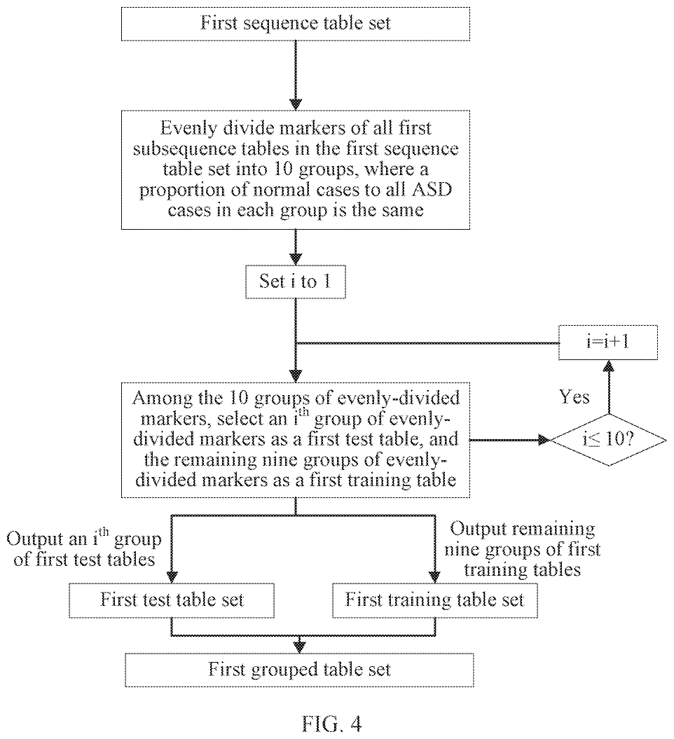

In this embodiment, as shown in FIG. 4, based on the preset table marker grouping condition, the stratified marker sampling is performed on all the first subsequence tables in the first sequence table set, and all the first subsequence tables are equally divided into 10 groups. In each group, a proportion of normal cases to all ASD cases is the same.

Specifically, in this embodiment, as shown in FIG. 4, i represents a group number, and each first subsequence table is divided into 10 groups. A first group of data in each subsequence table is used as a first test table, and the remaining nine groups of data are used as a first training table. Subsequently, a second group of data in each subsequence table is used as a first test table, and the remaining nine groups of data are used as a first training table. By analogy, a 10^{th} group of data in each subsequence table is used as a first test table, and the remaining nine groups of data are used as a first training table. All first training tables are combined to obtain the first training table set, and all first test tables are combined to obtain the first test table set. The first training table set and the first test table set are combined correspondingly to obtain the first grouped table set.

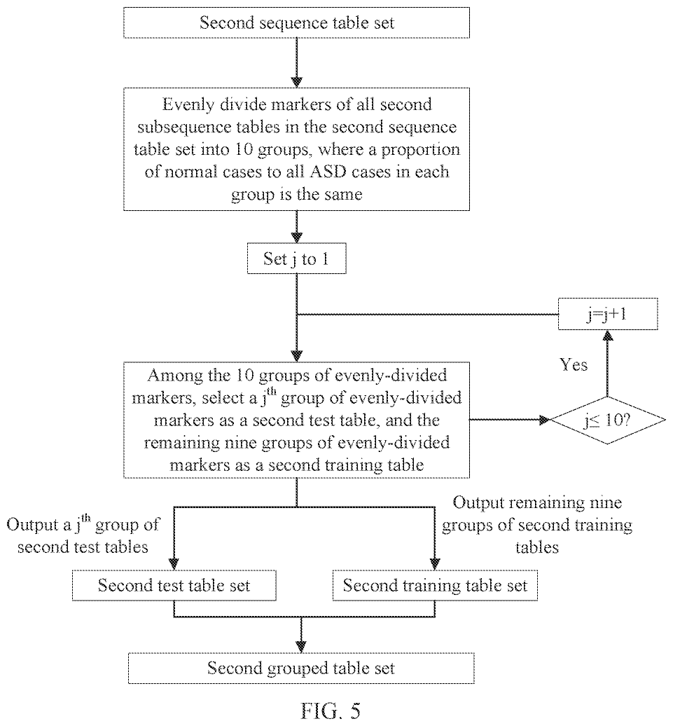

Similarly, specifically, in this embodiment, as shown in FIG. 5, j represents the group number, and each second subsequence table is divided into 10 groups. A first group of data in each subsequence table is used as a second test table, and the remaining nine groups of data are used as a second training table. Subsequently, a second group of data in each subsequence table is used as a second test table, and the remaining nine groups of data are used as a second training table. By analogy, a 10^{th} group of data in each subsequence table is used as a second test table, and the remaining nine groups of data are used as a second training table. All second training tables are combined to obtain the second training table set, and all second test tables are combined to obtain the second test table set. The second training table set and the second test table set are combined correspondingly to obtain the second grouped table set.

Step S103: Train and model the first training table set and the second training table set based on the random forest machine learning algorithm to obtain a first submodel set and a second submodel set respectively, import the first test table set into the first submodel set to obtain a first best characteristic combination, and import the second test table set into the second submodel set to obtain a second best characteristic combination.

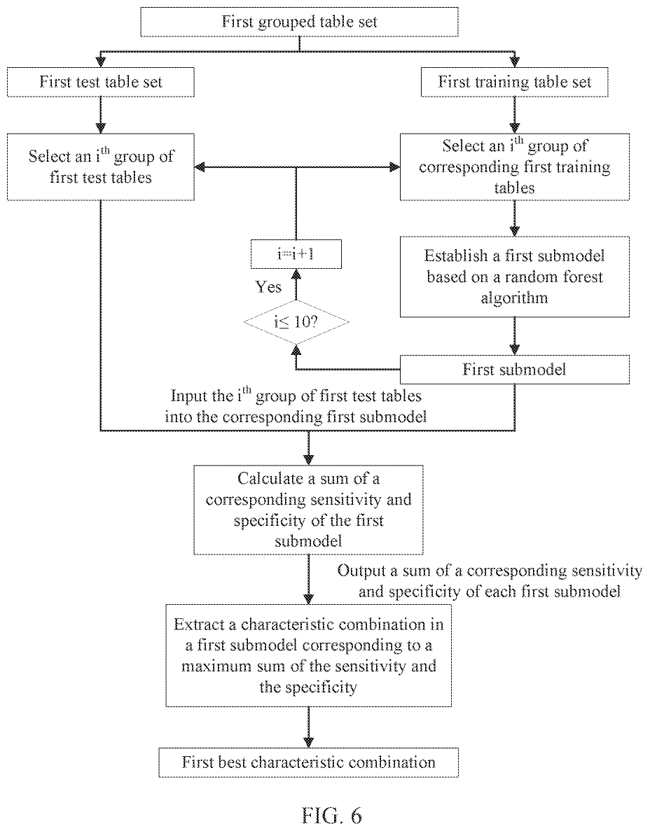

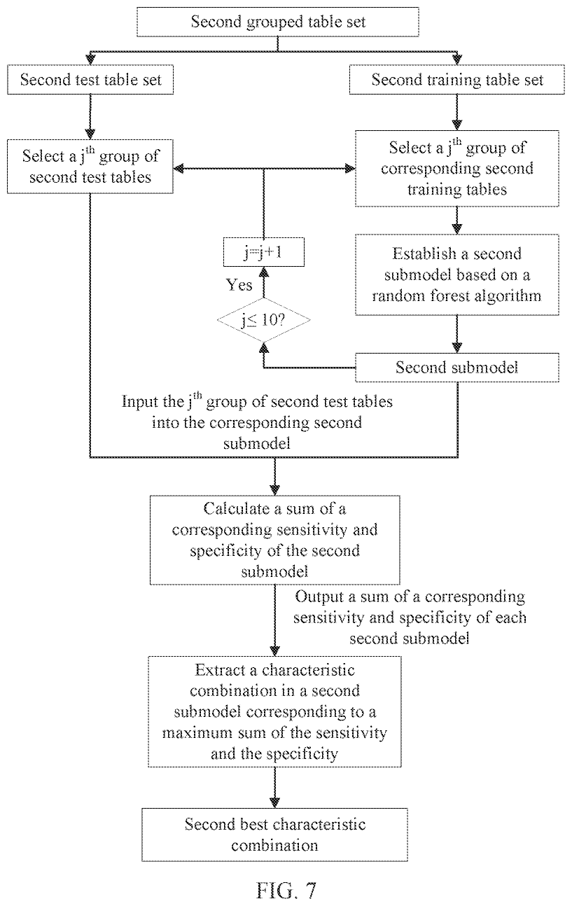

Preferably, as shown in FIG. 6 and FIG. 7, the first training table set and the second training table set are trained and modeled based on the random forest machine learning algorithm to obtain the first submodel set and the second submodel set respectively. Data of the first test table set is imported into the first submodel set to obtain a corresponding sensitivity and specificity of each first submodel, mean value summation is performed to obtain a characteristic combination in a first submodel corresponding to a maximum sum of the sensitivity and the specificity, and the obtained characteristic combination is taken as the first best characteristic combination.

In this embodiment, referring to FIG. 6, there are a total of 270 first submodels in the first submodel set (a total of 10 groups, with 27 first submodels in each group). Each submodel corresponds to a sum of one sensitivity and one specificity. Sums of sensitivities and specificities of the first training set and the first test set that belong to a same group are averaged, 27 averaged sums of the sensitivity and the specificity are compared, and the characteristic combination in the first submodel corresponding to the maximum sum of the sensitivity and the specificity is taken as the first best characteristic combination, in other words, a combination of 12 characteristics.

Similarly, preferably, data of the second test table set is imported into the second submodel set to obtain a corresponding sensitivity and specificity of each second submodel, mean value summation is performed to obtain a characteristic combination in a second submodel corresponding to a maximum sum of the sensitivity and the specificity, and the obtained characteristic combination is taken as the second best characteristic combination.

In this embodiment, referring to FIG. 7, there are a total of 270 second submodels in the second submodel set (a total of 10 groups, with 27 second submodels in each group). Each submodel corresponds to a sum of one sensitivity and one specificity. Sums of sensitivities and specificities of the second training set and the second test set that belong to a same group are averaged, 27 averaged sums of the sensitivity and the specificity are compared, and the characteristic combination in the second submodel corresponding to the maximum sum of the sensitivity and the specificity is taken as the second best characteristic combination, in other words, a combination of three characteristics.

Step S104: Obtain a first model based on the first best characteristic combination, stratified sampling of the first data table, and the random forest machine learning algorithm, obtain a second model based on the second best characteristic combination, stratified sampling of the second data table, and the random forest machine learning algorithm, and combine the first model and the second model to construct an ASD risk prediction model, so as to input a result of the ASD evaluation item into the ASD risk prediction model to obtain a prediction result.

It should be noted that the result of the ASD evaluation item is an ASD-related evaluation item. In specific implementation, for example, the result of the ASD evaluation item can be obtained based on a standardized questionnaire that is filled out by a parent based on an actual symptom of a child. A specific standardized questionnaire may be specified based on an actual usage requirement. The prediction result can be obtained by inputting the result of the ASD evaluation item into the ASD risk prediction model.

Preferably, based on the first best characteristic combination, the stratified sampling is performed on a characteristic that meets the first best characteristic combination in the first data table, and based on the random forest machine learning algorithm, iterative operation is performed on a first data table obtained after the stratified sampling to obtain the first model. Based on the second best characteristic combination, the stratified sampling is performed on a characteristic that meets the second best characteristic combination in the second data table, and based on the random forest machine learning algorithm, the iterative operation is performed on a second data table obtained after the stratified sampling to obtain the second model.

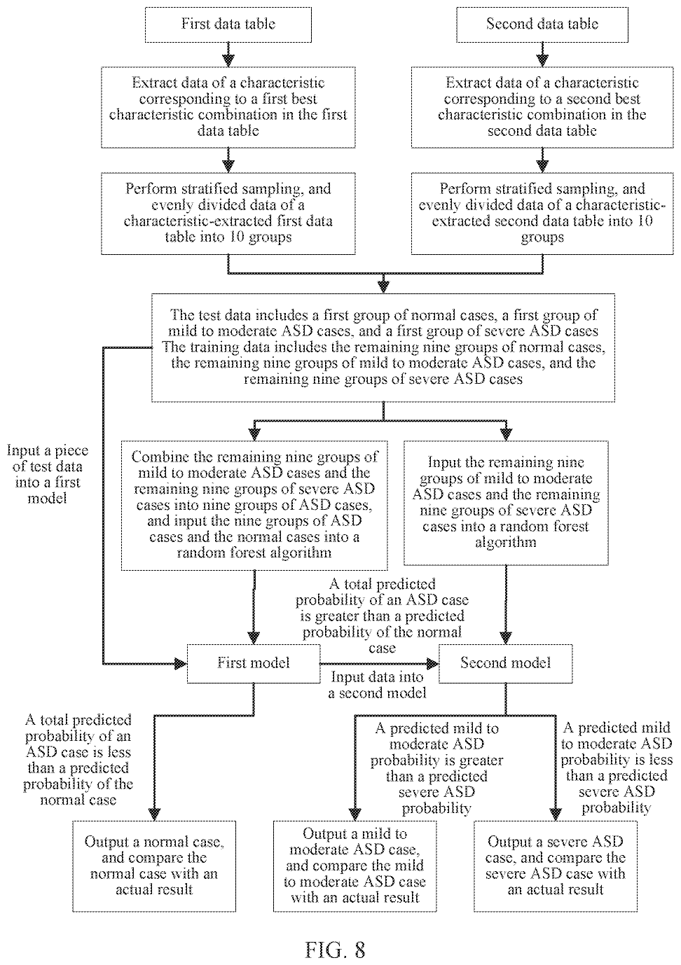

In this embodiment, referring to FIG. 8, based on the first best characteristic combination and the second best characteristic combination, the characteristic that meets the first best characteristic combination in the first data table, and the characteristic that meets the second best characteristic combination in the second data table are screened. The stratified sampling is performed on all markers in a screened first data table and a screened second data table, and all the markers are equally divided into 10 groups. Data of a first group of normal cases, a first group of mild to moderate ASD cases, and a first group of severe ASD cases is used as test data, while the remaining nine groups of normal cases, nine groups of mild to moderate ASD cases, and nine groups of severe ASD cases are used as training data.

In this embodiment, referring to FIG. 8, nine groups of mild to moderate ASD cases and nine groups of severe ASD cases are merged into nine groups of all ASD case data. Characteristic variables of the 12 characteristics in the first best characteristic combination are extracted from the nine groups of all ASD case data and nine groups of normal case data, and the extracted characteristic variables are input into the random forest machine learning algorithm to obtain the first model. Characteristic variables of the three characteristics in the second best characteristic combination are extracted from nine groups of mild to moderate ASD case data and nine groups of severe ASD case data, and the extracted characteristic variables are input into the random forest machine learning algorithm to obtain the second model.

In this embodiment, referring to FIG. 8, a combinatorial test is performed on the first model and the second model to construct the ASD risk prediction model, so as to input the result of the ASD evaluation item into the ASD risk prediction model to obtain the prediction result. Preferably, one test sample is extracted from the first data table obtained after the stratified sampling and the second data table obtained after the stratified sampling, and data information that meets the first best characteristic combination in the test sample is input into the first model to obtain a first predicted probability of the test sample. The first predicted probability includes a total predicted probability of an ASD case and a predicted probability of the normal case.

If the total predicted probability of the ASD case is less than the predicted probability of the normal case, it is determined that the test sample is a normal case; or if the total predicted probability of the ASD case is greater than the predicted probability of the normal case, data information that meets the second best characteristic combination in the test sample is input into the second model to obtain a second predicted probability of the test sample. The second predicted probability includes a predicted probability of the mild to moderate ASD case and a predicted probability of the severe ASD case.

If the predicted probability of the mild to moderate ASD case is greater than the predicted probability of the severe ASD case, it is determined that the test sample is a mild to moderate ASD case; or if the predicted probability of the mild to moderate ASD case is less than the predicted probability of the severe ASD case, it is determined that the test sample is a severe ASD case.

If the determining result is consistent with an actual situation of the test sample, the ASD risk prediction model is constructed, so as to input the result of the ASD evaluation item into the ASD risk prediction model to obtain the prediction result.

In this embodiment, referring to FIG. 8, the test sample includes the first group of normal cases, the first group of mild to moderate ASD cases, and the first group of severe ASD cases. For a test sample, a characteristic variable that meets the 12 characteristics in the first best characteristic combination is screened out, and then input into the first model to obtain a first predicted probability of the test sample. If a predicted probability of a predicted ASD case is less than the predicted probability of the normal case, the test sample is a normal case. If the predicted probability of the predicted ASD case is greater than the predicted probability of the normal case, a characteristic variable that meets the three characteristics in the second best characteristic combination is screened out, and then input into the second model to obtain a second predicted probability of the test sample. If the predicted probability of the mild to moderate ASD case is greater than the predicted probability of the severe ASD case, a model prediction result of the sample indicates that the sample is a mild to moderate ASD case. If the predicted probability of the mild to moderate ASD case is less than the predicted probability of the severe ASD cases, it indicates that the test sample is a severe ASD case.

In another embodiment, the step S104 is repeatedly performed. Data from a second group of normal cases, a second group of mild to moderate ASD cases, and a second group of severe ASD cases is used as the test data, and the remaining nine groups of normal cases, the remaining nine groups of mild to moderate ASD cases, and the remaining nine groups of severe ASD cases are used as the training data. By analogy, data from a 10^{th} group of normal cases, a 10^{th} group of mild to moderate ASD cases, and a 10^{th} group of severe ASD cases are used as the test data, and the remaining nine groups of normal cases, the remaining nine groups of mild to moderate ASD cases, and the remaining nine groups of severe ASD cases are used as the training data. When this embodiment is executed, 10 ASD risk prediction models consisting of the first model and the second model are generated, and sensitivities and specificities of the 10 ASD risk prediction models are averaged as an overall sensitivity and specificity of the model, in other words, overall performance of the model. For a severe ASD case, the sensitivity is 0.71, and the specificity is 0.95. For a mild to moderate ASD case, the sensitivity is 0.76, and the specificity is 0.90. For a normal case, the sensitivity is 0.94, and the specificity is 0.91. Overall confusion matrices of the 10 models are calculated and added up to obtain an overall confusion matrix A of the model.

$$A = ( \begin{matrix}28 & 12 & 0 \\ 8 & 54 & 10 \\ 3 & 6 & 130\end{matrix} )$$

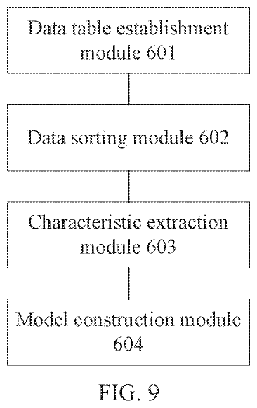

In addition, referring to FIG. 9, the present disclosure further provides a device for constructing an ASD risk prediction model, including: a data table establishment module 601, a data sorting module 602, a characteristic extraction module 603, and a model construction module 604.

The data table establishment module 601 is configured to establish a first data table and a second data table based on case information of a sample set. The sample set includes a sample of a mild to moderate ASD case, a sample of a severe ASD case, and a sample of a normal case. The first data table records case information of the sample of the normal case and case information of samples of all ASD cases. The second data table records case information of the sample of the mild to moderate ASD case and case information of the sample of the severe ASD case. Each piece of case information includes a characteristic, a characteristic variable, and a marker.

The data sorting module 602 is configured to perform characteristic arrangement and marker grouping on the first data table and the second data table according to a preset characteristic arrangement rule and marker grouping rule to obtain a first grouped table set and a second grouped table set respectively, where the first grouped table set includes a first test table set and a first training table set, and the second grouped table set includes a second test table set and a second training table set.

The characteristic extraction module 603 is configured to train and model the first training table set and the second training table set based on a random forest machine learning algorithm to obtain a first submodel set and a second submodel set respectively, import the first test table set into the first submodel set to obtain a first best characteristic combination, and import the second test table set into the second submodel set to obtain a second best characteristic combination.

The model construction module 604 is configured to: obtain a first model based on the first best characteristic combination, stratified sampling of the first data table, and the random forest machine learning algorithm, obtain a second model based on the second best characteristic combination, stratified sampling of the second data table, and the random forest machine learning algorithm, and combine the first model and the second model to construct an ASD risk prediction model, so as to input a result of an ASD evaluation item into the ASD risk prediction model to obtain a prediction result.

Preferably, that the characteristic arrangement and marker grouping are performed on the first data table and the second data table according to the preset characteristic arrangement rule and marker grouping rule to obtain the first grouped table set and the second grouped table set respectively specifically includes the following operations:
calculating a weight of each characteristic in the data table based on a classification accuracy rate, sorting the corresponding characteristic based on the weight of each characteristic, and performing characteristic extraction and addition on a characteristic-sorted first data table and a characteristic-sorted second data table to obtain a first sequence table set and a second sequence table set respectively, where the performing characteristic extraction and addition on a characteristic-sorted first data table and a characteristic-sorted second data table specifically includes: extracting the first two characteristics from the characteristic-sorted first data table and the characteristic-sorted second data table based on a characteristic arrangement order to form a first subsequence table and a second subsequence table respectively, then sequentially adding a next characteristic to the first subsequence table and the second subsequence table based on the characteristic arrangement order until all characteristics in the first data table and the second data table are added, to obtain a plurality of first subsequence tables and a plurality of second subsequence tables respectively, and combining the plurality of first subsequence tables and the plurality of second subsequence tables to obtain the first sequence table set and the second sequence table set respectively.

Further, stratified marker sampling is performed on all first subsequence tables in the first sequence table set and all second subsequence tables in the second sequence table set based on a preset table marker grouping condition and a same proportion of evenly divided markers to obtain the first grouped table set and the second grouped table set respectively.

Further, the first training table set and the second training table set are trained and modeled based on the random forest machine learning algorithm to obtain the first submodel set and the second submodel set respectively, the first test table set is imported into the first submodel set to obtain the first best characteristic combination, and the second test table set is imported into the second submodel set to obtain the second best characteristic combination. Specifically, the first training table set and the second training table set are trained and modeled based on the random forest machine learning algorithm to obtain the first submodel set and the second submodel set respectively; the first test table set data is imported into the first submodel set to obtain a corresponding sensitivity and specificity of each first submodel, mean value summation is performed to obtain a characteristic combination in a first submodel corresponding to a maximum sum of the sensitivity and the specificity, and the obtained characteristic combination is taken as the first best characteristic combination; and the second test table set data is imported into the second submodel set to obtain a corresponding sensitivity and specificity of each second submodel, mean value summation is performed to obtain a characteristic combination in a second submodel corresponding to a maximum sum of the sensitivity and the specificity, and the obtained characteristic combination is taken as the second best characteristic combination.

In this embodiment of the present disclosure, the data table establishment module 601, the data sorting module 602, the characteristic extraction module 603, and the model construction module 604 each may be one or more processors, controllers or chips that each have a communication interface, can realize a communication protocol, and may further include a memory, a related interface and system transmission bus, and the like if necessary. The processor, controller or chip executes program-related code to realize a corresponding function. In an alternative solution, the data table establishment module 601, the data sorting module 602, the characteristic extraction module 603, and the model construction module 604 share an integrated chip or share devices such as a processor, a controller and a memory. The shared processor, controller or chip executes program-related codes to implement corresponding functions.

The embodiments of the present disclosure have the following effects:

The embodiments of the present disclosure provide a method and device for constructing an ASD risk prediction model, which can further optimize and process information of a predicted ASD item more accurately. A data table is established, such that a large number of evaluation items can be called more accurately. Data sorting and characteristic extraction further improve the accuracy of a prediction result. Steps of model construction are optimized, and the model construction involves iteration, which can ensure that each piece of data can be accurately predicted in a random forest machine learning algorithm, improving convenience of the model construction and accuracy of model prediction.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A method for constructing an autism spectrum disorder (ASD) risk prediction model, comprising:
establishing a first data table and a second data table based on case information of an ASD sample set, wherein the sample set comprises a sample of a mild to moderate ASD case, a sample of a severe ASD case, and a sample of a normal case, the first data table records case information of the sample of the normal case and case information of samples of all ASD cases, the second data table records case information of the sample of the mild to moderate ASD case and case information of the sample of the severe ASD case, and each piece of case information comprises a characteristic, a characteristic variable, and a marker;
performing characteristic arrangement and marker grouping on the first data table and the second data table according to a preset characteristic arrangement rule and marker grouping rule to obtain a first grouped table set and a second grouped table set respectively, wherein the first grouped table set comprises a first test table set and a first training table set, the second grouped table set comprises a second test table set and a second training table set;
calculating a weight value of each characteristic in the data table according to a preset characteristic weight calculation method, sorting the corresponding characteristic based on the weight value of each characteristic, and performing characteristic extraction and addition on a characteristic-sorted first data table and a characteristic-sorted second data table to obtain a first sequence table set and a second sequence table set respectively, wherein the performing characteristic extraction and addition on a characteristic-sorted first data table and a characteristic-sorted second data table specifically comprises: extracting the first two characteristics from the characteristic-sorted first data table and the characteristic-sorted second data table based on a characteristic arrangement order to form a first subsequence table and a second subsequence table respectively, then sequentially adding a next characteristic to the first subsequence table and the second subsequence table based on the characteristic arrangement order until all characteristics in the first data table and the second data table are added, to obtain a plurality of first subsequence tables and a plurality of second subsequence tables respectively, and combining the plurality of first subsequence tables and the plurality of second subsequence tables to obtain the first sequence table set and the second sequence table set respectively; and performing stratified marker sampling on all first subsequence tables in the first sequence table set and all second subsequence tables in the second sequence table set based on a preset table marker grouping condition and a same proportion of evenly divided markers to obtain the first grouped table set and the second grouped table set respectively;
training and modeling the first training table set and the second training table set based on a random forest machine learning algorithm to obtain a first submodel set and a second submodel set respectively, importing the first test table set into the first submodel set to obtain a first best characteristic combination, and importing the second test table set into the second submodel set to obtain a second best characteristic combination;
obtaining a first model based on the first best characteristic combination, stratified sampling of the first data table, and the random forest machine learning algorithm, and obtaining a second model based on the second best characteristic combination, stratified sampling of the second data table, and the random forest machine learning algorithm, which specifically comprises:
performing, based on the first best characteristic combination, the stratified sampling on a characteristic that meets the first best characteristic combination in the first data table, and performing, based on the random forest machine learning algorithm, iterative operation on a first data table obtained after the stratified sampling to obtain the first model; and performing, based on the second best characteristic combination, the stratified sampling on a characteristic that meets the second best characteristic combination in the second data table, and performing, based on the random forest machine learning algorithm, the iterative operation on a second data table obtained after the stratified sampling to obtain the second model; and
combining the first model and the second model to construct an ASD risk prediction model, so as to input a result of an ASD evaluation item into the ASD risk prediction model to obtain a prediction result.

2. The method for constructing an ASD risk prediction model according to claim 1, wherein the establishing a first data table and a second data table based on case information of a sample set specifically comprises:
based on the sample of the mild to moderate ASD case, the sample of the severe ASD case, and the sample of the normal case in the sample set, collecting and preprocessing data information of the ASD evaluation item, extracting a characteristic, a characteristic variable, and a marker of the sample, screening out a common characteristic variable, calculating a score of each characteristic variable in ASD test indicator data information according to a preset scoring method, screening out a characteristic variable that can reflect a score of the ASD test indicator data information, and establishing the first data table and the second data table.

3. The method for constructing an ASD risk prediction model according to claim 2, wherein the training and modeling the first training table set and the second training table set based on a random forest machine learning algorithm to obtain a first submodel set and a second submodel set respectively, importing the first test table set into the first submodel set to obtain a first best characteristic combination, and importing the second test table set into the second submodel set to obtain a second best characteristic combination specifically comprises:
training and modeling the first training table set and the second training table set based on the random forest machine learning algorithm to obtain the first submodel set and the second submodel set respectively;
importing data of the first test table set into the first submodel set to obtain a corresponding sensitivity and specificity of each first submodel, performing mean value summation to obtain a characteristic combination in a first submodel corresponding to a maximum sum of the sensitivity and the specificity, and taking the obtained characteristic combination as the first best characteristic combination; and
importing data of the second test table set into the second submodel set to obtain a corresponding sensitivity and specificity of each second submodel, performing mean value summation to obtain a characteristic combination in a second submodel corresponding to a maximum sum of the sensitivity and the specificity, and taking the obtained characteristic combination as the second best characteristic combination.

4. The method for constructing an ASD risk prediction model according to claim 3, wherein the combining the first model and the second model to construct an ASD risk prediction model, so as to input a result of an ASD evaluation item into the ASD risk prediction model to obtain a prediction result specifically comprises:
extracting one test sample from the first data table obtained after the stratified sampling and the second data table obtained after the stratified sampling, and inputting data information that meets the first best characteristic combination in the test sample into the first model to obtain a first predicted probability of the test sample, wherein the first predicted probability comprises a total predicted probability of an ASD case and a predicted probability of the normal case;
if the total predicted probability of the ASD case is less than the predicted probability of the normal case, determining that the test sample is a normal case; or if the total predicted probability of the ASD case is greater than the predicted probability of the normal case, inputting data information that meets the second best characteristic combination in the test sample into the second model to obtain a second predicted probability of the test sample, wherein the second predicted probability comprises a predicted probability of the mild to moderate ASD case and a predicted probability of the severe ASD case;
if the predicted probability of the mild to moderate ASD case is greater than the predicted probability of the severe ASD case, determining that the test sample is a mild to moderate ASD case; or if the predicted probability of the mild to moderate ASD case is less than the predicted probability of the severe ASD case, determining that the test sample is a severe ASD case; and
if the determining result is consistent with an actual situation of the test sample, combining the first model and the second model to construct the ASD risk prediction model, so as to input the result of the ASD evaluation item into the ASD risk prediction model to obtain the prediction result.

5. A device for constructing an ASD risk prediction model, comprising: a data table establishment module, a data sorting module, a characteristic extraction module, and a model construction module, wherein
the data table establishment module is configured to establish a first data table and a second data table based on case information of a sample set, wherein the sample set comprises a sample of a mild to moderate ASD case, a sample of a severe ASD case, and a sample of a normal case, the first data table records case information of the sample of the normal case and case information of samples of all ASD cases, the second data table records case information of the sample of the mild to moderate ASD case and case information of the sample of the severe ASD case, and each piece of case information comprises a characteristic, a characteristic variable, and a marker;
the data sorting module is configured to perform characteristic arrangement and marker grouping on the first data table and the second data table according to a preset characteristic arrangement rule and marker grouping rule to obtain a first grouped table set and a second grouped table set respectively, wherein the first grouped table set comprises a first test table set and a first training table set, the second grouped table set comprises a second test table set and a second training table set, and the data sorting module is specifically configured to perform following operations:
calculating a weight value of each characteristic in the data table according to a preset characteristic weight calculation method, sorting the corresponding characteristic based on the weight value of each characteristic, and performing characteristic extraction and addition on a characteristic-sorted first data table and a characteristic-sorted second data table to obtain a first sequence table set and a second sequence table set respectively, wherein the performing characteristic extraction and addition on a characteristic-sorted first data table and a characteristic-sorted second data table specifically comprises: extracting the first two characteristics from the characteristic-sorted first data table and the characteristic-sorted second data table based on a characteristic arrangement order to form a first subsequence table and a second subsequence table respectively, then sequentially adding a next characteristic to the first subsequence table and the second subsequence table based on the characteristic arrangement order until all characteristics in the first data table and the second data table are added, to obtain a plurality of first subsequence tables and a plurality of second subsequence tables respectively, and combining the plurality of first subsequence tables and the plurality of second subsequence tables to obtain the first sequence table set and the second sequence table set respectively; and performing stratified marker sampling on all first subsequence tables in the first sequence table set and all second subsequence tables in the second sequence table set based on a preset table marker grouping condition and a same proportion of evenly divided markers to obtain the first grouped table set and the second grouped table set respectively;
the characteristic extraction module is configured to train and model the first training table set and the second training table set based on a random forest machine learning algorithm to obtain a first submodel set and a second submodel set respectively, import the first test table set into the first submodel set to obtain a first best characteristic combination, and import the second test table set into the second submodel set to obtain a second best characteristic combination; and
the model construction module is configured to: obtain a first model based on the first best characteristic combination, stratified sampling of the first data table, and the random forest machine learning algorithm, obtain a second model based on the second best characteristic combination, stratified sampling of the second data table, and the random forest machine learning algorithm, and combine the first model and the second model to construct an ASD risk prediction model, so as to input a result of an ASD evaluation item into the ASD risk prediction model to obtain a prediction result, wherein
the obtaining a first model based on the first best characteristic combination, stratified sampling of the first data table, and the random forest machine learning algorithm, and obtaining a second model based on the second best characteristic combination, stratified sampling of the second data table, and the random forest machine learning algorithm specifically comprises: performing, based on the first best characteristic combination, the stratified sampling on a characteristic that meets the first best characteristic combination in the first data table, and performing, based on the random forest machine learning algorithm, iterative operation on a first data table obtained after the stratified sampling to obtain the first model; and performing, based on the second best characteristic combination, the stratified sampling on a characteristic that meets the second best characteristic combination in the second data table, and performing, based on the random forest machine learning algorithm, the iterative operation on a second data table obtained after the stratified sampling to obtain the second model.

6. The device for constructing an ASD risk prediction model according to claim 5, wherein the training and modeling the first training table set and the second training table set based on a random forest machine learning algorithm to obtain a first submodel set and a second submodel set respectively, importing the first test table set into the first submodel set to obtain a first best characteristic combination, and importing the second test table set into the second submodel set to obtain a second best characteristic combination specifically comprises:
training and modeling the first training table set and the second training table set based on the random forest machine learning algorithm to obtain the first submodel set and the second submodel set respectively;
importing data of the first test table set into the first submodel set to obtain a corresponding sensitivity and specificity of each first submodel, performing mean value summation to obtain a characteristic combination in a first submodel corresponding to a maximum sum of the sensitivity and the specificity, and taking the obtained characteristic combination as the first best characteristic combination; and
importing data of the second test table set into the second submodel set to obtain a corresponding sensitivity and specificity of each second submodel, performing mean value summation to obtain a characteristic combination in a second submodel corresponding to a maximum sum of the sensitivity and the specificity, and taking the obtained characteristic combination as the second best characteristic combination.
